(19) United States
(12) Patent Application Publication
(10) Pub. No.: US 2026/0159555 A1
(43) Pub. Date: Jun. 11, 2026

(54) NOVEL O-ANTIGEN VIBRIO PARAHAEMOLYTICUS AND APPLICATION THEREOF

(71) Applicant: ZHEJIANG UNIVERSITY, HANGZHOU (CN)

(72) Inventors: YU CHEN, HANGZHOU (CN); XIAO CHEN, HANGZHOU (CN); RUONAN WANG, HANGZHOU (CN); QI WANG, HANGZHOU (CN)

(21) Appl. No.: 19/413,552

(22) Filed: Dec. 9, 2025

(30) Foreign Application Priority Data
Dec. 10, 2024 (CN) 202411808329.0
Oct. 31, 2025 (CN) 202511579578.1

(51) Int. Cl. C07K 14/28; A61K 39/00

(52) U.S. Cl. CPC C07K 14/28; A61K 39/00

(57) ABSTRACT

The invention discloses a novel O-antigen of Vibrio parahaemolyticus, a somatic antigen of Vibrio parahaemolyticus being named O17, Vibrio parahaemolyticus being classified as Vibrio parahaemolyticus O17:K4 with a preservation number of CGMCC No. 31706, a DNA sequence of the somatic antigen O17 of the vibrio parahaemolyticus being shown in SEQ ID NO:1.

### CROSS REFERENCE TO RELATED SEQUENCE LISTING

This application contains a computer readable form of a Sequence Listing, the name of the file being “Sequence”, created on 26 Feb. 2026 and electronically submitted via Patent Center on 26 Feb. 2026. The size of the xml file are 35,482 bytes, which are incorporated herein by reference.

### FIELD OF THE INVENTION

The invention belongs to the field of biomedicine, relates to a novel somatic antigen and a preparation method for a specific serum therefor, and in particular, to the preparation of a novel O-antigen of Vibrio parahaemolyticus and a specific serum therefor. The mycelium is Vibrio parahaemolyticus.

### BACKGROUND OF THE INVENTION

Vibrio parahaemolyticus (VP) is a major pathogen causing foodborne illnesses. Humans are mainly infected through exposure to seawater, or by consuming raw or undercooked seafood. It usually causes acute gastroenteritis, and occasionally wound infections and sepsis, which can lead to death in severe cases. Data from our country's foodborne disease outbreak surveillance network shows that in coastal areas, Vibrio parahaemolyticus has surpassed Salmonella and Staphylococcus aureus to become the most important biological factor in foodborne disease outbreaks.

Serotyping of Vibrio parahaemolyticus is of great significance in epidemiological studies and outbreak monitoring. Currently, most research on Vibrio parahaemolyticus worldwide uses serotype as the proper term for international communication to distinguish different biological phenotypes of Vibrio parahaemolyticus. Vibrio parahaemolyticus can be serotyped based on the heat-stable somatic lipopolysaccharide O-antigen and the heat-unstable capsular polysaccharide antigen. There are 16 types of O-antigen and more than 70 types of K-antigen. The identification is mainly performed by slide agglutirlation. With the advancement of molecular biology techniques, whole-genome sequencing technology has been gradually applied to the molecular typing of Vibrio parahaemolyticus in recent years, achieving a resolution down to the base level for strains. Based on whole-genome sequencing, complete bacterial genome sequences can be obtained, and the O/K-antigen gene clusters of Vibrio parahaemolyticus can be analyzed to predict the serotype. Prior to 1996, Vibrio parahaemolyticus infection did not show a clear dominant serotype. Until February 1996, a novel O3:K6 serotype strain was discovered that caused a mass food poisoning outbreak in Kolkata, India. This newly-emerged strain subsequently spread rapidly across continents among populations in many coastal countries and regions around the world, leading to several large-scale outbreaks and becoming the most prevalent pathogenic type of Vibrio parahaemolyticus. Recent reports show that a new serotype of Vibrio parahaemolyticus, O10:K4, was first detected in Guangxi, China in 2020. This strain exhibits characteristics of a pandemic strain and has caused multiple outbreaks and sporadic food poisoning incidents in the area. Then, reports emerged in Beijing, Zhejiang, Guangxi, and other regions that O10:K4 Vibrio parahaemolyticus had become the most prevalent serotype in those areas. According to data from 2013 to 2022 from National Foodborne Disease Surveillance Point System, the newly emerging O10:K4 serotype of Vibrio parahaemolyticus in 2020 has replaced O3:K6 as the current dominant circulating serotype. Our research team selected 100 strains of O10:K4 Vibrio parahaemolyticus from different years between 2020 and 2023 for whole-genome sequencing and analyzed their O-antigen gene clusters. We found that the O-antigenic determinant of this serotype strain contains 17 genes with a total length of 16030 bp. The known O10-antigenic determinant contains 16 genes with a total length of 14977 bp, sharing 9621 identical bases with O17, representing a similarity of 60% (9621/16030). Furthermore, this new O-antigenic determinant is different from the sequences of the other 15 O-antigenic determinants besides O10, and it is closest to the O4-antigenic determinant, with a similarity of 82%. Therefore, it is believed that this O-antigen determinant is a new type, which can be named O17. The existing literature reports on O10:K4 Vibrio parahaemolyticus, and the serotyping reagents used are all internationally recognized commercial reagents-Denka Seiken, Tokyo, Japan. This diagnostic serum kit contains O-serum (O1-O11) of 11 clinically common Vibrio parahaemolyticus strains. We hypothesize that the O10-serum in existing commercial reagents cross-agglutinates with Vibrio parahaemolyticus containing the novel O17 somatic antigen, thus leading to inappropriate results.

The invention relates to a novel somatic antigen O17 of Vibrio parahaemolyticus and a preparation method for a specific serum therefor, which can effectively diagnose and surveillance the prevalence and outbreak of Vibrio parahaemolyticus of this serotype, and is of great significance for the prevention, control and treatment of related diseases.

### BRIEF SUMMARY OF THE DISCLOSURE

An objective of the invention is to provide a novel O-antigen of Vibrio parahaemolyticus, is and the mycelium is Vibrio parahaemolyticus; the somatic antigen is named O17, the vibrio parahaemolyticus is classified as Vibrio parahaemolyticus O17:K4 with a preservation number of CGMCC No. 31706, and a DNA sequence of the novel somatic antigen O17 of the vibrio parahaemolyticus is shown in SEQ ID NO:1.

### Determination of Novel Somatic Antigen O17 Strains Provided by the Invention

Vibrio parahaemolyticus is identified using matrix-assisted laser desorption/ionization time-of-flight (MALDI-TOF) mass spectrometry or biochemical reactions. Serotyping is performed using diagnostic serum for Vibrio parahaemolyticus from Denka Seiken, Tokyo, Japan (containing 11 O and 69 K). The strain is identified as Vibrio parahaemolyticus serotype O10:K4 strain by slide agglutination. Whole-genome sequencing is performed on 100 strains of O10:K4 Vibrio parahaemolyticus, and the O-antigenic determinant located in the dgkA and hldD regions of chromosome 1 is analyzed to find that the O antigen determinant contains 17 genes (see FIG. 1, numbered 00267-00283), with a total length of 16030 bp (see SEQ ID NO:1). The known antigenic determinant of O10 contains 16 genes (see FIG. 1), with a total length of 14977 bp, which shares 9621 identical bases with O17, and the similarity is 60% (9621/16030) (see FIG. 2). The new O-antigenic determinant is different from the sequences of the other 15 O-antigenic determinants besides O10 (see FIG. 2), and is closest to the O4-antigenic determinant, with a similarity of 82% while missing 6 genes compared to O4 but having inserted with 2 new genes. The genes at both ends of the novel O-antigen determinant are relatively conserved and exist in multiple known O1-O16 antigen determinants. The 00273 and 00275 genes (located in the sequence intervals of 6284-7204 and 8147-9814, respectively, see SEQ ID NO:1) are specific genes for the antigen determinant (see FIG. 3). According to the annotation, both specific genes 00273 and 00275 encode proteins homologous to transposases of the insertion sequence family. Therefore, it is believed that the O-antigen determinant is a new somatic antigen type of Vibrio parahaemolyticus, which may be named O17.

Three strains with complete genome sequences are selected and compared with a Vibrio parahaemolyticus strain (GenBank accession number: CP102433-4) that appears in Thailand and is identified as serotype 010:K4. The selected strains have an additional 16557 bp sequence containing 13 genes on chromosome 1. Among the 13 genes, 9 encode putative proteins, 1 encodes a chromosome segregating protein (Smc), and 2 encode tyrosine recombinases (XerC) (see SEQ ID NO:2).

The strain of Vibrio parahaemolyticus is preserved on Oct. 31, 2024, at the China General Microbiological Culture Collection Center (No. 3, Courtyard 1, Beichen West Road, Chaoyang District, Beijing, Institute of Microbiology, Chinese Academy of Sciences), with preservation number of CGMCC No. 31706 and the classification name recommended by the collection center is Vibrio parahaemolyticus O17:K4.

Another objective of the present invention is to provide a preparation method for the novel Vibrio parahaemolyticus strain antigen O17 serum, which is achieved through the following steps:

\textcircled{1} Antigen Preparation: Suspend the Vibrio parahaemolyticus strain containing antigen O17 in a solution of 3% NaCl+5% glycerol and subject it to high-pressure inactivation. Wash twice with PBS and collect the sediment by centrifugation. Resuspend the bacterial body in a 0.9% saline solution, emulsify by adding Freund's adjuvant at a volume ratio of 1:1 to obtain the immunogen.

\textcircled{2} Collection of Immune Serum: Inject New Zealand white rabbits (aged 9-12 weeks) with the immunogen (10\hat{ }CFU/mL), at six sites per rabbit (four needles on the back and two in the groin), 200 μL per site. Conduct a booster immunization two weeks after the first dose, followed by weekly immunizations (with the same dosage each time). After five immunizations, collect whole blood, place it at 37° C. for 2 hours, then overnight at 4° C., centrifuge, and the supernatant is the immune serum.

\textcircled{3} Preparation of Specific Immune Serum: Perform agglutination reactions between the immune serum and both the novel Vibrio parahaemolyticus strain antigen O17 and other known O serotype Vibrio parahaemolyticus strains. In case of cross-reaction, adsorb the immune serum overnight at 4° C. successively with other known O serotype Vibrio parahaemolyticus strains that have undergone high-pressure sterilization treatment (suspended in 3% NaCl+5% glycerol solution, subjected to high-pressure inactivation, and washed twice with PBS). Centrifuge and the supernatant is the specific immune serum for the novel strain antigen O17.

\textcircled{4} Performance Verification and Application of Specific Immune Serum: The new O17 serum exhibits strong specificity, showing significant agglutination with the novel Vibrio parahaemolyticus strain antigen O17, but not with nine other known O antigens of Vibrio parahaemolyticus strains or with other enteropathogenic bacteria such as Vibrio cholerae (non-O1/O139 serogroups), Salmonella enteritidis, Shigella dysenteriae, Shigella flexneri, and Aeromonas schubertii. No agglutination occurs with gut-colonizing bacteria including Escherichia coli, Klebsiella pneumoniae, and Enterobacter cloacae.

The novel O17-serum exhibits good sensitivity; even after serial dilution to 64 times with physiological saline, the serum still shows significant agglutination with the O17 strain.

The novel somatic antigen O17-serum for Vibrio parahaemolyticus prepared by the invention has high specificity and can be used for the identification of the strain of the serotype, which is of great significance for the diagnosis and surveillance of Vibrio parahaemolyticus in foodborne diseases and infectious diarrhea.

With the novel O17 serum provided by the invention, slide agglutination experiments are performed on Vibrio parahaemolyticus from patients with acute diarrhea in clinical settings, and a total of 253 novel somatic antigen O17 strains are identified. 100 strains are randomly selected for whole-genome sequencing, and the O-antigenic determinant is analyzed. The sequencing results show that the antigenic determinants are all O-antigenic determinants, and are consistent is with the serum agglutination results.

### Advantages and Effects:

1. The advantage of the invention is that the O-antigenic determinant type of the novel somatic antigen (O17) of Vibrio parahaemolyticus is clarified, and in particular, the specific gene that distinguishes the antigenic determinant from other O antigenic determinants is clarified.

2. The advantage of the invention is that a preparation method for a specific serum of Vibrio parahaemolyticus based on the clarified O-antigen type is provided. The novel O17-serum has high specificity and may overcome the defect of cross-agglutination of Vibrio parahaemolyticus of the novel O17 somatic antigen against O10-serum caused by existing Denka Seiken, Tokyo, Japan's Vibrio parahaemolyticus diagnostic serum (Denka Seiken, Tokyo, Japan).

3. The advantage of the invention is that a novel somatic antigen (O17) of Vibrio parahaemolyticus and a preparation method for a specific serum therefor are provided, which may be conveniently and quickly used to detect a novel O-serotype Vibrio parahaemolyticus that has become the most prevalent strain in recent years. The invention provides important detection technologies for the diagnosis, surveillance and control of pathogens causing foodborne diseases and infectious diarrhea.

### BRIEF DESCRIPTION OF THE DRAWINGS

FIG. 1 shows the gene distribution of the novel O17-antigenic determinant and the gene similarity comparison with the O10- and O4-antigenic determinants.

FIG. 2 shows the similarity comparison results between the sequence of the novel O17-antigenic determinant and the sequences of 16 known O-antigens.

FIG. 3 shows the distribution of 17 genes of the novel O17-antigenic determinant in 16 known O-antigenic determinants.

FIG. 4 shows the agglutination reaction between the immune serum of the novel somatic antigen O17 for Vibrio parahaemolyticus and strains O17 and O10.

FIG. 5 shows the agglutination reaction of the novel O17-specific immune serum with 10 known O-serotype strains.

FIG. 6 shows the application of the novel O17-specific immune serum.

### DETAILED DESCRIPTION OF THE INVENTION

In order that the objectives, technical schemes and advantages of the present invention will become more apparent, the present invention will be described in more detail with reference to the embodiments. It should be understood that the specific embodiments described herein are only for illustrating but not for limiting the present invention.

It should be noted that the following detailed descriptions are exemplary and are intended to provide further explanation of the invention. Unless defined otherwise, all technical and scientific terms used herein have the same meaning as commonly understood by those skilled in the art to which the invention belongs.

It should be noted that the terms used herein are for describing particular embodiments only and are not intended to limit the exemplary embodiments according to the invention. As used herein, unless the context clearly indicates otherwise, the singular forms are intended to include the plural forms as well. Furthermore, it should be understood that when the terms “comprises” and/or “includes” are used in this specification, they specify the presence of features, steps, operations and/or combinations thereof.

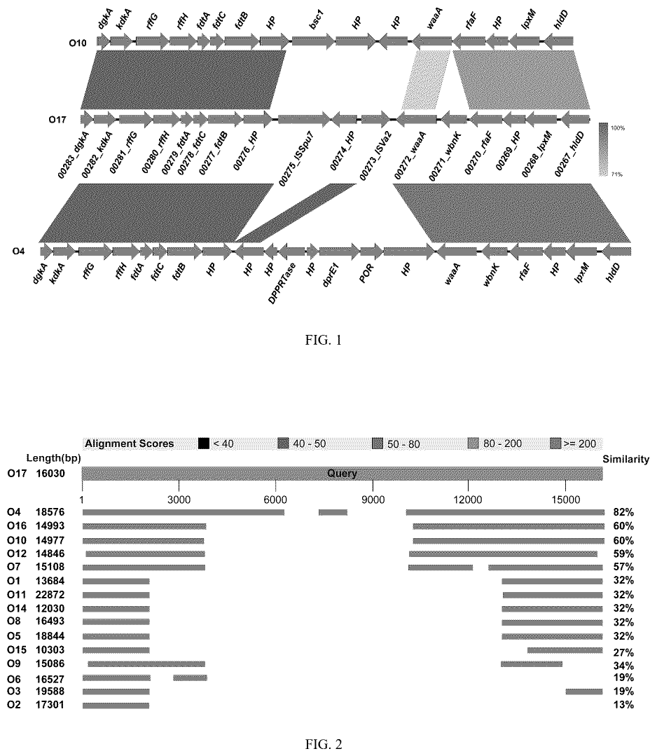

FIG. 1 shows the gene distribution of the novel O17-antigenic determinant and the gene similarity comparison with the O10- and O4-antigenic determinants. The O-antigen gene cluster of Vibrio parahaemolyticus is located between the dgkA and hldD genes. Grayscale shading indicates the degree of sequence similarity, and arrows denote the direction of gene transcription.

FIG. 3 shows the distribution of 17 genes of the novel O17-antigenic determinant in 16 known O-antigenic determinants.

Genes designated as 00273 and 00275 are unique to the O17 antigenic determinant and are absent from all previously characterized O1-O16 antigen clusters.

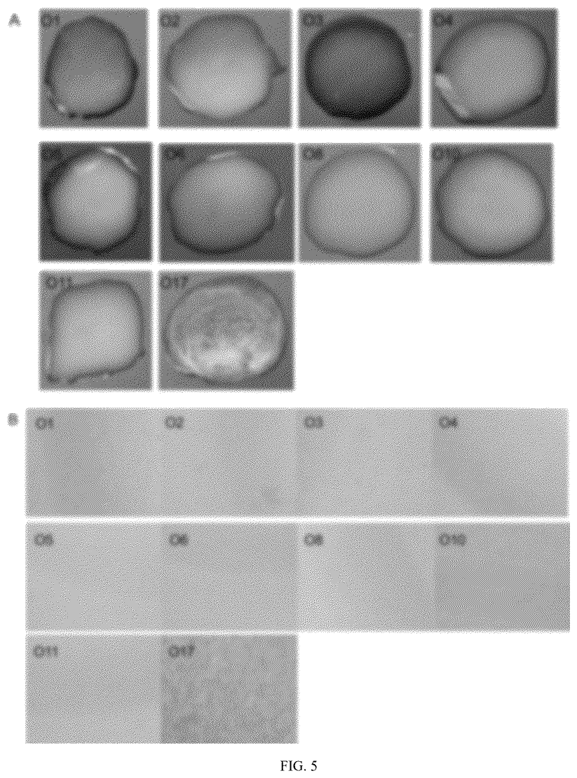

FIG. 4 depicts agglutination reactions between the immune serum raised against the novel Vibrio parahaemolyticus strain expressing O17 somatic antigen and strains of O17 and O10 serotypes. In panel A, the left side shows no agglutination between the novel O17 strain and saline (negative control), while the right side demonstrates clear agglutination between the same O17 strain and O17-specific immune serum. In panel B, the left side shows no agglutination between the O10 strain and saline (negative control), whereas the right side reveals cross-agglutination between the O10 strain and the O17 immune serum.

FIG. 5 illustrates agglutination reactions of the novel O17-specific immune serum with ten known O-serotype strains. Panel A shows macroscopic agglutination patterns between the O17-specific serum and various O-antigen strains; panel B shows microscopic agglutination of the same combinations.

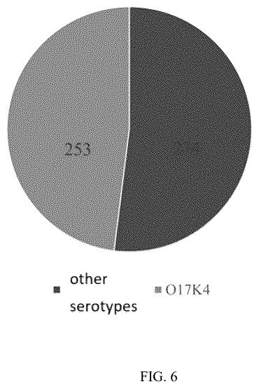

FIG. 6 demonstrates an application of the novel O17-specific immune serum. Using this serum in slide agglutination assays, 253 out of 527 Vibrio parahaemolyticus isolates collected between 2019 and 2023 were identified as harboring the novel O17 somatic antigen (serotype O17:K4).

### Embodiment 1: Identification of a Novel Somatic Antigen (O17) Strain of Vibrio parahaemolyticus

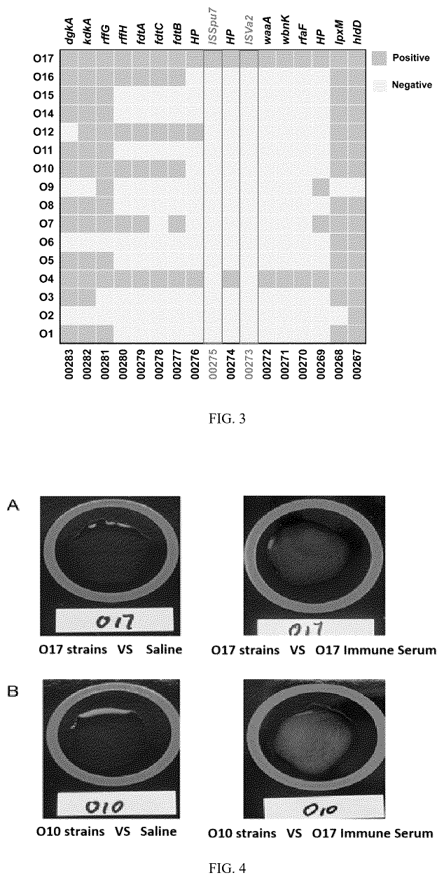

Fecal specimens from patients with acute diarrhea are inoculated into alkaline peptone water, enriched at 37° C. for 6 hours, and then transferred to TCBS medium. After incubation at 37° C. for 18 hours, a single colony is selected and identified as Vibrio parahaemolyticus by Bruker matrix-assisted laser desorption/ionization time-of-flight (MALDI-TOF) mass spectrometry. Using the diagnostic serum for Vibrio parahaemolyticus from Denka Seiken, Tokyo, Japan (containing 11 O and 69 K), with physiological saline as a negative control, the serotype of Vibrio parahaemolyticus is determined by slide agglutination. Whole-genome sequencing is performed on 200 strains of O10:K4 serotype Vibrio parahaemolyticus to analyze the O-antigenic determinants of the strains. The sequences are all identical (see SEQ ID NO:1). The O-antigenic determinant of the strain has 9621 identical bases to the known O10-antigenic determinant with a similarity of only 60% (9621/16030), is different from the sequences of the other 15 known O-antigenic determinants except O10 and is most similar to the O4-antigenic determinant with a similarity of 82, and has two specific genes (00273 located in the sequence interval 6284-7204, 00275 located in the sequence interval 8147-9814) that are not found in the other 16 O-antigenic determinants. The O-antigenic determinant may be named O17. The distribution of the 17 genes in the sequence of the novel O17-antigenic determinant and the comparison results with the O10-antigenic determinant are shown in FIG. 1. The similarity comparison results with the sequences of 16 known O-antigenic determinants are shown in FIG. 2. The distribution of the 17 genes contained therein in the other 16 O-antigenic determinants is shown in FIG. 3. It is evident that the O17 is a novel somatic antigen for Vibrio parahaemolyticus.

Further, three strains with complete genome sequences are selected and compared with a Vibrio parahaemolyticus strain (GenBank accession number: CP102433-4) that appears in Thailand and is identified as serotype 010:K4. The selected strains have an additional 16557 bp sequence containing 13 genes on chromosome 1. Among the 13 genes, 9 encode putative proteins, 1 encodes a chromosome segregating protein (Smc), and 2 encode tyrosine recombinases (XerC) (see SEQ ID NO: 2), clarifying that the selected strains are distinctly different from the Thai strains.

### Embodiment 2: Detailed process of preparation of a serum for the novel somatic antigen O17 of Vibrio parahaemolyticus

Preparation of antigen: The novel somatic antigen O17 for Vibrio parahaemolyticus is inoculated into BHI medium containing 3% NaCl and cultured overnight at 370° C.; then the mycelia are collected by centrifugation at 4500×g for 30 min, washed twice with PBS, resuspended in a solution of 3% NaCl+5% glycerol and autoclaved, washed twice with PBS again and then centrifuged at 4500×g for 30 min; the collected mycelia are resuspended in physiological saline solution for immunization of rabbits.

Immunization of rabbits: Three healthy New Zealand white rabbits that are 9-12 weeks old are selected to be allowed to adapt to the environment for 1-2 weeks; the somatic antigen (1010 CFU/mL) resuspended in 0.9% saline is emulsified with Freund's adjuvant at a 1:1 volume ratio; each rabbit is immunized at 6 sites (4 injections on the back and 2 injections in the groin), 200 μL per site. Two weeks after the initial immunization, a booster immunization is administered, followed by weekly immunizations with the same dosage as the initial immunization. After five immunizations, whole blood is collected via cardiac stenosis.

Collection of immune serum: After being collected and placed at 37° C. for 2 hours, then the whole blood is transferred to 4° C. overnight, and is centrifuged at 12000×g for 10 minutes on the next day to collect the supernatant, which is the immune serum.

Preparation of specific immune serum: The collected immune serum is subjected to slide agglutination with the novel somatic antigen O17 strain, wherein the specific steps for slide agglutination are as follows: the slide is divided into sections using a marker pen, and the prepared novel O17-immune serum of 1 drop (about 5 μL) is added to each section; physiological saline is added to one section as a control to exclude self-agglutination, and then, the bacterial suspension of 5 μL is added to each section, mixed thoroughly, and allowed to react for 2 minutes; finally, agglutination is observed. The collected immune serum shows very obvious agglutination with the novel somatic antigen O17 strain (FIG. 2A). Given that the novel somatic antigen O17 strain agglutinates with the diagnostic serum O10 of Vibrio parahaemolyticus from Denka Seiken, Tokyo, Japan (previous literature reported as 010:K4 strain), this study performs the slide agglutination test on the prepared O17 immune serum and the somatic antigen O10 of Vibrio parahaemolyticus. The results show that there is a cross-agglutination reaction between the O17-immune serum and the somatic antigen O10 strain (FIG. 2B). The somatic antigen O10 for Vibrio parahaemolyticus is selected and suspended in the solution of 3% NaCl+5% glycerol, autoclaved, washed twice with PBS and centrifuged to collect the precipitate so as to obtain the O10 somatic antigen. Freshly-prepared O10 somatic antigen is used to adsorb the O17-immune serum overnight at 4° C. The supernatant obtained by centrifugation at 12000×g for 10 min is the specific immune serum (O17-serum) for the novel somatic antigen O17 of Vibrio parahaemolyticus.

### Embodiment 3: Performance of Novel O17 Specific Serum

The prepared O17-specific serum of Vibrio parahaemolyticus may rapidly detect the novel O17 serotype strain through agglutination with the novel somatic antigen O17 strain, does not agglutinate with any of Vibrio parahaemolyticus of 9 known O serotypes (FIG. 3, Table 1), does not agglutinate with other enteropathogenic bacteria such as Vibrio cholerae (non-010139 group), Salmonella typhimurium, Salmonella enteritidis, Shigella dysenteriae, Shigella flexneri and Vibrio fluvialis (Table 2), and does not agglutinate with intestinal colonizing bacteria Escherichia coli, Klebsiella pneumoniae and Enterobacter cloacae (Table 2). The O17-serum is serially diluted with physiological saline, and is still obviously agglutinated with the novel somatic antigen O17 strain when being diluted to 64 times (Table 3). The novel O17-serum exhibits excellent specificity and sensitivity.

TABLE 1
Agglutination of novel O17-specific serum with
Vibrio parahaemolyticus of different O serotypes

| | Vibrio | |
|---|---|---|
| | parahaemolyticus of | |
| | different O serotypes | Agglutination |
| 1 | O1 | Not agglutinated |
| 2 | O2 | Not agglutinated |
| 3 | O3 | Not agglutinated |
| 4 | O4 | Not agglutinated |
| 5 | O5 | Not agglutinated |
| 6 | O6 | Not agglutinated |
| 7 | O8 | Not agglutinated |
| 8 | O10 | Not agglutinated |
| 9 | O11 | Not agglutinated |
| 10 | O17 | Agglutinated |

TABLE 2
Agglutination of novel O17-specific serum
with other enteropathogenic bacteria

| | Bacteria | Agglutination |
|---|---|---|
| 1 | Vibrio cholerae | Not agglutinated |
| | (non-O₁O₁₃₉ group) | |
| 2 | Salmonella typhimurium | Not agglutinated |
| 3 | Salmonella enteritidis | Not agglutinated |
| 4 | Shigella dysenteriae | Not agglutinated |
| 5 | Shigella flexneri | Not agglutinated |
| 6 | Vibrio fluvialis | Not agglutinated |
| 7 | Escherichia coli | Not agglutinated |
| 8 | Klebsiella pneumoniae | Not agglutinated |
| 9 | Enterobacter cloacae | Not agglutinated |

TABLE 3
Serial dilution of novel O17-specific serum and agglutination
with O17 somatic antigen for Vibrio parahaemolyticus

| | Agglutination with O17 |
|---|---|
| | somatic antigen of |
| | Vibrio parahaemolyticus |
| | (10¹⁰ CFU/mL) |
| Dilution of serum at 1:1 | Not agglutinated |
| before immunization | |
| Immune serum stock | Agglutinated |
| solution | |
| Dilution of novel O17 | Agglutinated |
| specific serum at 1:2 | |
| Dilution of novel O17 | Agglutinated |
| specific serum at 1:4 | |
| Dilution of novel O17 | Agglutinated |
| specific serum at 1:8 | |
| Dilution of novel O17 | Agglutinated |
| specific serum at 1:16 | |
| Dilution of novel O17 | Agglutinated |
| specific serum at 1:32 | |
| Dilution of novel O17 | Agglutinated |
| specific serum at 1:64 | |
| Dilution of novel O17 | Not agglutinated |
| specific serum at 1:128 | |

### Embodiment 4: Application of Novel O17-Specific Serum

The inventors have long conducted research on the variation and evolution of Vibrio parahaemolyticus from patients with acute infectious diarrhea, and have established a Vibrio parahaemolyticus strain library. Using the prepared novel O17-specific serum, a slide agglutination experiment is performed. Among a total of 527 Vibrio parahaemolyticus strains from 2019 to 2023, 253 strains of Vibrio parahaemolyticus with the novel somatic antigen O17 (O17:K4) have been identified (FIG. 4). Whole genome sequencing is performed on 100 of these strains, and the O antigen determinant is analyzed. The results show that all of them are novel O17-antigen determinants, i.e., all of them are novel somatic antigen O17 of Vibrio parahaemolyticus. If the novel O17-specific serum provided by the invention is not used, identification is currently only possible through bacterial whole-genome sequencing. Whole genome sequencing is costly and requires a 3-4 week testing cycle, making it unsuitable for routine clinical laboratory use. Therefore, the novel O17-specific serum may conveniently and quickly identify the novel somatic antigen O17 of Vibrio parahaemolyticus.

1. A novel O-antigen of Vibrio parahaemolyticus, a somatic antigen of Vibrio parahaemolyticus being named O17, Vibrio parahaemolyticus being classified as Vibrio parahaemolyticus O17:K4 with a preservation number of CGMCC No. 31706, a DNA sequence of the somatic antigen O17 of the vibrio parahaemolyticus being shown in SEQ ID NO:1.

2. Two specific genes of the somatic antigen O17 according to claim 1 are located in intervals of sequences 6284-7204 and 8147-9814 respectively.

3. The specific genes according to claim 2 are 00273 and 00275 genes.

4. A preparation method for a serum of the novel O-antigen O17 of Vibrio parahaemolyticus according to claim 1, comprising:
\textcircled{1} preparation of antigen:
suspending a Vibrio parahaemolyticus strain of the somatic antigen O17 in a solution of 3% NaCl+5% glycerol for inactivating by high pressure; washing twice with PBS, centrifuging and collecting a precipitate; resuspending the mycelium in a 0.9% physiological saline solution, emulsifying with Freund's adjuvant at a 1:1 volume ratio to obtain an immune antigen;
\textcircled{2} collection of immune serum:
injecting New Zealand white rabbits aged 9-12 weeks with immune antigens of 1010 CFU/ml with 200 μL injected into each rabbit at six sites, and administering a booster immunization two weeks after an initial immunization, followed by weekly immunizations with the same dose each time; after 5 immunizations, collecting whole blood for incubating at 37° C. for 2 hours, then at 4° C. overnight and centrifuging to obtain a supernatant as the immune serum;
\textcircled{3} preparation of specific immune serum:
subjecting the immune serum to an agglutination reaction with the somatic antigen O17 of Vibrio parahaemolyticus and other known O-serotype Vibrio parahaemolyticus, and if cross-reactivity occurs, using the known O-serotype Vibrio parahaemolyticus that have been autoclaved to adsorb the immune serum overnight at 40° C. and centrifuged to obtain a supernatant as the specific immune serum for the somatic antigen O17 strain.

5. The preparation method according to claim 2, wherein the autoclaving is performed by suspending the strain in the solution of 3% NaCl+5% glycerol, inactivating by high pressure, and washing twice with PBS.


SEQUENCE LISTING (1 sequence)

SEQ ID NO: 1 - DNA, 16030 bp, Vibrio parahaemolyticus
  source 1..16030: mol_type = genomic DNA
gatcatcgtaactggtggcgctggcatgatcggcagcaacattgttaaagcactaaacgaagctggtattaacgatatcttggttgtcgacaacctaaaaaatggcaaaaagtttaagaacttagttgatctggacatcacagattacatggatcgtgacgactttctgacccaaattatggcgggcgatgattttggtccgattgaggcaattttccacgaaggcgcttgctctgcaacgacagagtgggatggtaaatacatgatgctaaacaattacgagtactctaaagagctgcttcattactgtttagatcgtgaaattccattcctatacgcctcttccgcagcaacttacggcgatacagatactttcgttgaagaacgcgaatacgaaggcgcactaaatgtatacggttactcgaaacagcagtttgataactacgttcgtcgtctatggcaagacgctgaagaacatggtgaaaccctttctcaaatcaccggcttccgttacttcaacgtatatggcccacgtgaagatcacaaaggcagcatggcatctgttgcgttccatctgaacaaccaactaaacgcaggtgaaaatccaaaactgtttgcaggtagcgagtcttttaagcgtgatttcgtttacgttggtgatgtatgtaaagtaaacctgtggttccttgaaaatggtgtttctggcatcttcaactgtggtactggtcgtgcagagtcttttgaagaagtggcaaaagcagttattcaacaccacaacaaaggtgaaatccaaaccattccgttccctgagcacctaaaaggtgcttaccaagaattcactcaagcggacctaaccaagcttcgtgcagctggttgtgatgtagaattcaaaacggtagctgaaggtgttgctgaatacctagcgattcaaaaccgttaattcccttcaaaggtggtagttaactctctaccacctttactctgatacgattactagtatcatttctacagacccccaatcgcctttcttcaaaaatataggtacttaaaacttcatgactaatcaacgtgatgatttcgatcctaaagcttataaccctgaatttgagcgccactttttagcgccgaagtattgggggacgtggataggcgttgtcctttctttgccgcttgctttactacctttaggggttcaaaaatggctagcaaaaactatcgcgaagaaactggctaaatcgcacaagggtccagctcaccgagctcgagtcaactttgagctgtgcttcccagagaaatcatttgaagagcgcgaagcactgattgaaaaaacgctttataccgcagcattgttctttttccgtttcagcttacttagcttacgttcatctaaatggttacagcagcgttgccagatccgaggtttagacatccttcaaaaacacatcgacaatgatgagaaagtcattttgatggttccacactcatgggctatcgatgttcctgctattttgctggcttcaatggggatgcctgtttctgcgatggcaaaaaaacaaaagaatgatgtggctgactggttaatgcacaagcaaagagttcagtatggaggccgagtttacgagcgctctggtggaattaagccttttctaaaatctattcgagacggctacattgggtattacctacccgatcaagatcatggccctgaacacagcgtatttgtcgactttttcgccacacgtaaagcaacgctccctggattgggtaaaattgctaaactatcacgttccaaagtattacctctgttcgcttcaatgaacaccgaagatggcacattcgatatcgaaattttgcctgcatttgatttagacaaaggtgaagaacaagacgcccgcacctgtaacgaagctattgaatacttcgtcggtgataaaccagaacaatatatgtgggtgttgcagcttttacactctcaagaagatagcaataattattacaatttgtttaaaccaagatataacagcgactggaaagtgcagtgaaagtatttgtcgtaagcttggctcgatcactagatcgccgagaacgtattgaagaaaagttaaaacaacagggcattgagtttgaattttttgatgccgtagatggatctattgacggttttcttcattcagaaaaaagccgtccagccataactcagcgccgcaaagggtatcaattaaaaaccagtgaagtcgcttgcttctcaagccattacgagttgtgggttcgctgtactgagctcgatgaaccgatcattatcttagaagataacgtcgaccctgtagaaggattaaagcaagtactcgcgcatacccttccgctgacttctacttatgactacatcaagctgtctgccactcagaagcgtgtattcactccaatcataccgattgatgatagtcatatgctaggaggctactctgctgggacttgtggtaccaccgcatatattgtcacaccgaaagcagcctctaagtttgttaagcatgctcaagagtttatcgaaccagtagatgactacatggaaaaaccatggcgtcataatgttcagacttacagtgttgccccagatcttttcactagagctcaaatcgcgtcaacgattggcgctaaaagaaaagacaagtcgaagatgaccgtacttaacaagatctacgcagaactcttcagaacctacgaatccattcttaagtttatgtactggaagaacaaagcatgaaaatactcatcatcggaccgtcttgggttggtgatatggtgatgtcacaatcactgtacatcgttttaaagcaacttcatccagaaagtcaaattgatgtaattgcacctggttggtgtaaaccaattcttgagcgcatgccagagatccaccaagccattgaaatgcctattggtcatggtgagttcaacttattaggtcgtcgagaaattggtaaatcattacgagaaaaacagtacgaccacgcctacatcctgccgacgtctgcaaagtcagcgctgattccttggtttgctaatatcccacttcgcactggttggaaaggcgagatgcgctacggtctgctcaatgacttgcgcccaaatatgaaatctttccagttcatggttgagcgctatgttgcattagcctattcacaagcggagatgatcgactcttcttctctgggcggcttagaaacattacctcgccctagcctgtttattgataaacaagagcaaggtgccacgatttctaagtttaatctaaaccaagaatctcctgttgtgggtctatgccctggtgcggaattcggtcccgcgaaaaaatggcccgaagcgcactatgcagaagtggcaattcaaatggcaaaatctggacatcaggtatggctgtttggctctcaaaaagatctcgaaacctgcaataacatcaagcaattagtaccacaagaacatcagcatcagattcacgttctagcagggcaaacaagcctgattgaagcggttgatcttctggcggcttgccagaccgttgttgcgaacgactcaggcctgatgcacgtggcagcggccgttggctgtaacgtcgtggcggtctatggctccacctcaccgaaatacacgccaccacttgcagagaaggtagtgattgttcatacggacattgactgccgtccatgctttaagcgcgagtgtcagtttaagcatttgaagtgcttaactgagttatctccagaaaaagttctaaacagtattcagaagctcgagtcaaatagaataaaatacaaataaaaaatggaaaggaataaatcttgaaaatagtcaacgttgttggtggtttaggaagtcaaatgttcgcatatgctctttaccttagtttaaaggaaagattcggcgatcaagaatctatcctattagatacttctttctaccgaaaaaacaaacaacataacgggtatgagttagaaagattatttaacatcaaagagcaacctaacaacctctttaccaaaggtatattgttgaatagaggttttgttttcaaaaagttacgtaaactcctacttaaacgtttagatgcagcagataacaactataacttcgatccgaacgtatttaacaatgaaagtacgctctatattcaatcatggacgtcttggaagtatttctctgattgcactgatttagtccaatcagccttcacttttaaaccattaaaagatgaaaaaaacattgagatatcgaacataattaccagtgttaactctgtttccattcatgttcgtcgaggggattacctcaattctccagcattatcaggcttagctccactttggtattatcgcaatgcaatcaaacatcttgaagaaaacacaaacaacccaacctattttgttttttctgatgatatccaatggtgtcgagataacctagctctatcaaatgctcattatattgactggaatacaggagaggatagctacaaagacatccagctaatgagtctctgcaaacacaatattattccaaatagcagttttagttggtggggcgccttcctaaatgaaaattcggacaaaattgtcatcgcacctgaaacgtggggcaataagtcagtcggagttgaactaaaggatatgaattaccctagttggctggtaattccaaatactgaaaagtaaatattatcgtatcaatgcgactctctaagccgagtcgcattttagaccttcatttcgatgtaagtgataaactagtaatattctactcatcctcagcaaactgaacactctcaacgccatggttatcaaatgtttatcagaattacatacactctattactgacgcttgtatcaccaatttttttatttagtctatacaaaaaaaaaccaaacaaacctaggtttggtccgcgctggaaggaacattttggcgtaacgccacgattaaagtcgacgaataagcctatttggatccatgcagtatcagtgggagagtgtattgcagcaacgccactcatcgaagctctaaaaaagcaaaccccagagcaaactattttagtaacaaccactactagcaccggagctgaacaaatatcaaagctaggtgaacttgttgaacatcgctatatgcctatcgactttagctttgctgttaaagggtttttaaagaaaatacagccaaagcaaatgttaatcatagaaaccgagctttggccaaataccttaagcgaggttcacaaagcaggaataccaataactgtagttaatgcacgcttatctgcgaagtcatgtaataactataccagagttcagccgctatttaacttgatgcaaccttgcatcaccaaagtactttgccaaacggaatcagatgcagctcgatttgaacacctggggctagacagagagaaactgtcgataacaggttcgattaagtttgatattcaaatatcgggagacataagagaaaaaagtaaaaaattaagacaagcatttggcaaaagccgccctgtatggatagctgcaagcactcataaaggggaagatgagcaagtactaatagcacatcaacaagtattaaagtcacatccgaatgcacttttgatccttgtacctcgccaccctgaacgctttgacagtataaatgaactatgccaaaagtggggctttgagactgtacgtcgaacaacacacacgagtgtaactgaatcgactcaagtttatcttggagatacgatgggggaaatgctcttattgttaggtgtatctgacgtatgttttatgggtggaagcttagttggagacaaagttggagggcacaatatgttagagccggctgctttgggtatcccaaccattactggtcccagctacttcaattttaaagaagtagctgaaaacttaattgctctaggagcgttaaggcagattcaagataaacgagaactcgctcatgcccttactcagataattcaaaatccggacacctccaaagagatggctcgaaatgcagaagtattcgtgaaaaaaaaccagggcgctatagcgaaaacgataaattttttgttggatatatcccccagagactaaacgtctgggggagagaggaatagttatctaccttatcttatacacatcaaaatactgtcctctttcaacaggattatattgattaatgacataatcactaactgttgaataaaactggctttgttgcgtaattcagacgagaaaccaaacctcctaaagttgagcgacttttatacaatatactgagtttcaggcataccaagccctgtaagtttgttcagtgctttgatcatggcgtaagtctcgccaacctgagcgttgtaatttcttagacttaatttcccacctagcaactgttttactcggtacattgcggtctctgacagagagcgcttatgatagccataccgctttttccacttcttgttggagccgtagagcttctggcaacctaccgctaagttacgagggtgcccttgttcccagaaggctgctccttctcttggcgggatgagcggaacagctcgcttgatccgtatggcatcatggcaattccttgtgtcataagcgccatcgcctgatatctcaatgattttacgacgtgtttgcttgagtaagttggggagcacttcggcatcagtgacgttagataagctcagctctgctgcgactatttcgtgggtgcttgtatctaccgcgatatgcagcttacgccagactcttcgcttcccatcagttccatgcttcttgaccttccattcaccttcgccataaaccttgaggccagtggcatcaatggccagatgttgtattgctcctctggttttggttttaaatgaaacttcaacttccttagctcggcggcttatacaggtgtagtgcgggcaaacaagcgggatgttagccagcttaaatacggagtctagaaaaccttgcagcgctctcaagggcatagagaaaacgcgtttcaccatcagtgcagtagtaatggctaagtcgctgaatcggcgaggtctaccacgcttgccttgtttgttttgcttccactcagctatcgcttcctcatcaatccaaaaggtcagagaaccacggttgattaaggatttattgtactgcttccagttagttgttttgtaacgaggtttaggcatgaggctacgacgattgatgggcgtagccgctcagatcgtagtttcttgatttagttccatcgaattacgcaacaaagccggtatcgcctcatacaaatcattaaatgaaccaacaacgcttacattaataaaattcaataaaatggaaaaagtcgatattgcatttattttatttatgctttcatcgagctatataacgagagctaccagatggttattctttatacaacaacacccacaggaaagtaaatccacactatttcattttgcagtttatctatctggttttgcttttacaacaacacctggaaaatctggagagttagctcgatctatttacttaacgcctataggtgttccatttaattatacatttgcatgttttatttcagaaagatctttagatttagcagtagttggattgatttcttctattgcattttatcaattagactttcactttatctggactgctttattagcattacctctcatattcttagttgttatttttttaactaagaaattaataacaaagaaaatcaccttattcccatatcatgttttgacatacatattgcaactatttgattcaaaaaaagcaccttggccagttttattatcttttgtcgcctggatatctcaaggtctgattttagtaaagttttcaaactcattaaacattgaattatctactatcactttaattagtatatactgctcaggccttattattggagcaatatcactaataccagcaggaataggggcaacggagttaggcattgtatttttattaacaagctttggtgtcaataatcaagatgctttcgcgatatccgttgccagtagagcgataaccttattacctgcgctaagtctaggactactctgttcaataatacttaccacaaaaaataaaaccgagattccgcagccaatttaggaataaactgcttcatacagaacaccaagaagcttaagcagccgagtctgatgctcttgaacacccgttatgaagctttggccgccaaattcgagtgtgtcgataccttcgaattttaagaatacccaacgtgccgtcggtttagttgtcgtcttgttctttaccatgctaggaaagaattcctcggtcttagtgagactctctcggattttgtgctctaagcttgcgtagacgagtaggctcagcgtcatgatcatcagtagtgcctcgattcgctgaggtttcttcaggaaaattgatgatgttaagaactcagggcttttcaaaaagcgaaagccgcgttcgaccttttgctgagacttgtaatgttcaagcaatgcaaccattgttaggtcggtgttgtcagtatcgttggtggcgagaataaacatacctactttaagcttggccagtttcaccttttctagatcggtataaggcgtagcatctatcaaatagtggtatcccgtcggcttttcatctttcttcggccgaccacgacctgaataaattggctctttaacgatggttgattgcgcgaagccaagcaaatgacactggttggcgaagtcgctcattgcttgctcggcatccactgcgcaagcaaactgctttttctttagttgtcccagcgctttcagctctttggtgatatttttgtctaagttcttataaaacgttacctgttctcgcttggttgcttgttcactatgaatcaacagccacttctgagataccgagccataatcagcgtcaacccaacaacccgagtagccattgccgatatggctcagctgctctggctcaaggttcattaaggcctctttggctgatttaatggtcattggaacacgcgtaataaacttctgttgttgctcatccagtgaacgaatgctctcttcggtgtataaagcggcatcggcgatgaagtagcgactgttttgagctgccttcaggcaatgaatatgccgtttagtgacttctgagaatgccttagcatcattggtgttaccactaagcgcttgcatgtaaacaggcaggccagcttgattttcgcaaatcaactcaaggaccacttgatttagctctggacggtggtctcggctgtagccttttaccagcttgatggcattaagatcttcgtcctgggcgtattcaccatcgacatggaagctagtgatatcaaggtgtacagagtctgttttaagtcctagcttatcaacgacatgctctgcaatcgcttgataaagtgcggagacatctgcttcatataaagcatctaacgtgcgtccgagtacatcgtccgttaaatggtgagcttcaatatctttagcaagcaacttggcaacaggcttcgtctcgaagaagtcagaaaacatatgcagcgttctgctgtgaaagccaagaccattgagaatcatcgccaagaccgcatcgccatgtgaaacgttatgatccgagtacttgggaatgacgcgatcaataataccaggtaagccgatttcatggcagaaagcagcgatcagccctaagtgatctaggcgtttaatgacaggttgagcagcagagataataaagtgaccgttaaataacagtaatagatcaaaactggggatcgcggtcaagaggtctgtcgcactaacatttgtaaacgatcactggatcacattgttttaattctgaaattggctgcggaatgacggataaaaaacttttcaatagtatttaatttataatcctatactaatcaaagtactctatctattttatctataacggacattaactattttagccttctaatcaccttaatggcagtttgatagtctctagtttctaaaaaatagtgtaaagaagagaaaatcagagctaacttttttgatggaatagatgacataaactcagcagtacgcatccgttttttaatcgcactcacagcttcattggcatttttactctgcgcatctcgtaagttcattttaagatcttgcagctcacgctgagcattgatcactgtgttgttatatttttcttcgcgtgtttcttttgcgaacgttgatatgccacctagccgatattgaatccactttttgtgttcatgatgaataccatttaacaaaaaggctcggagaacattgatccggtcttcattgatgatgtctatgttgtagggctcaaactcattcaatacatcagaggtaaaagcagcacaacagccatctactgcacactttcgattaataatattttcgagtgtgtaaggttttttattttgcttgatcgattcaaatggctcaatgacatcaccttgctcagagattttgtcgtaactaccaataattaagcttggtttaccatttttcaaccaagtttcataagtttcctcaattcggttcggcatcgctatatcatcgccagcttgtacaataataagcttattttggtactgcccccgcaaggagttgatttggctaactaaaccaatatttttcttattgtggcgatattctatatttaatcctttaggcgcaaattctaaaagcgatagaatcttttcatgactatcatctggagagcaatcgtccaatactaacagcaggctcggccatgctgtttgctccaatgccgccggaagcgttttatcgagataacttgactgtttatagcaaaagaagacgtagacaacatcttccaatactcgaacctgatctttcataacgcctagaccttaaaagagtttacagcatcgaccacaacatttcgctcttgctcactcaagtatggtgacatcggcaatgacagcacctcattttgcaactgctccgtcagcggcaatgacagatgactaaactctttataagcctgctgtttatgcggcgctatcgggtaatgtacttgtgtctgaataccatgctctgataaatgcttcaccaatgcatctcggtttgaactgcgcaccacaaacaaatgccatacatgggcttcaggctcagccacttccggtaagataatattcgagttgttgatttcacgtaagtaaaattccgccagctcctgacgcgctttcgtctgcgctggaaggtagttcagtttcactgacaacatcgccgactgaatttcatccaaacgactgttcaatccttgataaagattctggtatttaacatgagagccgtagttacgaagggcacgcaaggtttctgccagttcatcatcgctagtcgtaatagcaccaccatcacctaacgcacctaggtttttacccggatagaagctaaaaccagaggcatccccccatgctccagacttcttaccagcaagtgtcgcaccatgagactgagcgctgtcttccagcacgagaaggttgtattttgtcgccaacgccataatcgctggcatatcggctaactgaccatacaaatgcaccggcaaaatcactttagtcttttctgtaatcgccgcttcaatcgcttcaggactgatattgtgcgtctgctcatccggctccacaaacaccggtaccaagtccgcttcggtgaccgctaatactgatgcgatataggtattgcccggtacgataacttcatcacctttgttaagtttgcctagctctcgccaagcctttaacgttagtgttaaagcatctaagccattcgcaacgcctacacagtgtttaactccacagtagtcagcaaatgcttgttcaaactgttccgtttcattaccaagaatgtaccagccagattcaatcacgcgtgcacaagcttgttttaattcgtcttcatactgcgcatttagacgcttaagctctaaaaaattaaccattatttttctctacataacgtactatcttggctggattcccaaccacaaccgcattgtccggcactgaacgggtgaccacactacccgctccaaccatcgcattttttccaatggttatgcctggcaaaatcgtggcattggcgccgatggaggcgcctgacagaatttttgttttcggaaatgcctctggccatacttttgaacgtgggaacttgtcgttcgtgaaagcagctgccgggccaatgaacacatcatcttcaatttcaatgccatcccaaagataaacgccgcattttacggtgactctatctcctaggaccacatcattctcgatgaaggtatgggcacagatattacagtcacgaccaattttagccccagcaaggaccacggcaaattgccaaatagacgtcccctcaccaatcgtcgatgaagcaacatcactgagtgcatgaattcgcatgggctacctctttaagaaatgtgtcatagtcacggatataatcggcttcatcatacaattcactagcgacaaccattagcacacaatcttcagaaaagtcgtgcatctcacgccattttaaccctttgatatgcaatcctacatcgggagaagaaagcgtcactgtctctttcgtggtgccatcatcaattaagaaagtcacgcaacctttcatacaaatagcgacttgctctaagtcgtagtgtgcatgaaaccctctcgctaaaccgctttgtgtatcaaagatgtagtaaatacgtttgatatcaaaaggaatgtttttatgttgctctaacgagattaatgagcctcgctcatcacccattttctgaaaagatattagcggtatattcgacatttattcttccaataactctagaagatagcggccataatcgtttttagataaggcttttcctgcggcttgaatgtgctctcgcgttaaccaccctcggcgatatgcaatctcttctaagcaagccactttataaccctggcgtttctcaatcgtttcaacaaaatgcgcagcttcaagcaaactgccatgcgtgccggtgtctaaccacgcaaagccgcgccctagtacgtcaaccgtcaacttcccttgctctaagtaagcttggttaatactcgtaatttctagctcgccacgatgggacggttgaatagttttggcaatgtccacgactcgattatcgtagaaatacagccctgtcacggcgtaattggattttggctcgattggcttctcttcaatcgatagtgctcgcatctcgtcgtcaaattccaccacaccaaagcgctcaggatctttcactttatagccaaagaccgtcgcaccttctgttaatgctgcggcttgctgtagcttcggcgaaaaaccttgcccataaaagatgttgtcacccaacaccaaacatactgagtcatcgccaatgaactcttcgccgatgatgaaggcttgagctaagccatctggacttggttgaatcgcgtattgcaactcaatgccaaactctgagccatcacccaataggcgctgaaatccttcctggtcttctggcgtggtgatgatcaagatttcacgaatgccagcaagcatgagtaccgacaacgggtagtaaatcattggtttgtcataaaccggcagtaactgcttagagacaccgcgtgtaatagggtaaaggcgagtgcccgagccacctgctaaaataatgcctttcattattcgcccgctcctaatcgctctaaactgtatgaaccatcaagcacgcgttgccaccattgcgggttgtttaggtaccattccacggttttacgaatgccgctttcaaaggtttcttctggcgtccagttaagctcttgagcaatcttggtcgcatcaattgcgtagcgcacatcatgacctgggcgatctttgacataagtgatcaaagattcataagactcaacacctgccggcttgtctggtcgcagctcttcaagcaaagcacaaatggttttcactacttcgatgtttgctttttcgttatggccaccgatgttgtaggtctcaccgatttcaccttccgtcacgactttgtaaagcgcgcgagcatggtcttccacaaacagccaatctcggatttgcatgccatcaccatacacaggtaaaggcttgccatcgagggcattcagaatcatcagtgggatcaacttctctgggaagtggtatgggccatagttgtttgagcaattggttacgagtgttgggagaccataagtacgctgccaagcgcgaaccaggtgatcgctcgacgctttagacgccgagtatgggctgcttggagcgtatgaggtggtttcggtgaataaatcatccgtaccctctaaatcgccatacacttcatccgtagaaatatgatgaaaacgaaacgccgatttgtccgcttcctcaaggtttgaccaatattgacgcgccgcttcaagaagatggtaagtgcccatgacgttggtctcaataaaagcagcaggaccatcaatagagcgatcgacatgcgactcagccgctaaatgcatcaccatatcgggttgatgctctgaaaacacgcggtctagttcagttcggtcacagatatccacttgctcgaagtaataacggtcactgtccgcgacatctaccaacgactccaagtttcctgcgtaggtgagcttgtctaaattcacgacgctatcttgggtgtcacgaataatgtgtcgaacgacagctgagccaatgaagcctgccccgcctgttactagtattttcattgcaaatgttacctactttaatgggtacttttagtgggtgtaccgaaatgcgctattggattgtctcacgaataactaacaaaaagctcactttacgtattttcagtatgacttttttgtacatgatcaaaatcggacgcttgccaaatgatttggcgtttgagcagttcttttttgaatgagcgaagaagcctttctaggttttgctttttccagtcgccgtgctcttgtttgcggcacttatcaaaatcaataatccacactttatctttgtcatcgattaagatgttgtggatattcaggtctgtgtgattaacgccagcgttgtgcattttggcaatttcttgccctattttttgatacattccttccggtaacggtttttcctgcaaaatactgaccaggtctcttgcgttaggaatgcgctcactcaataaatcggcctgatatgtcaatccagatttcacagctctcgcagcaattggccttggcacatgaacgccagcattaatcagtgtaagtaatagttggaactcttgcgcacaacgcgtttgctcccaacctgaaaaccaataattgtctttaactaacttaccaaacaagccaccacgtcgataatgacgcagagcggcttgcatcgtatcaagctgaacaaaccaagtcgtgccacgccctgacgcactgccagttactttgttggtggattgccaatactcggcatcaaaaataggctgaataggatcctcaataagctcctcatcaaaccaaatgacttggttactatctcgatactgctgaatcatgttgtaacctctagcctatgtagcgactggccaagatgtatagccaagaaaaagctgcgaacgctaacgcaaccataacggctgccgatcctatatctttagcgcgaccacttaattcatggcgttcaacaccaatgcgatccaccactgcttctatcgcggagttaatcagttcgacaatcaaaactaataccacgacaccaatcaataagatgcgctctatcatcccgacatcaaccacgcacgccaaaatcacagcaatgcttagcaaagcgagctcttgtcgaacagcagcttcatgtttaaaagcggcttttaatccctgtatagaataacctgtcgctttgataatgcgctttaccccagtattgcctggctttccagattcttgcgattgatgcggtgttgtctttgacattgca